(19) United States
(12) Patent Application Publication
(10) Pub. No.: US 2006/0039869 A1
(43) Pub. Date: Feb. 23, 2006

(54) Intranasal delivery of antipsychotic drugs

(76) Inventors: Daniel Wermeling, Lexington, KY (US); Jodi Miller, Acworth, GA (US)

Correspondence Address: MAYER, BROWN, ROWE & MAW LLP, P.O. BOX 2828, CHICAGO, IL, 60690-2828, US

(21) Appl. No.: 10/920,153

(22) Filed: Aug. 17, 2004

(51) Int. Cl. A61K 9/14; A61K 31/445; A61L 9/04

(52) U.S. Cl. 424/46; 514/317

(57) ABSTRACT

An intranasal drug product is provided including an antipsychotic drug, such as haloperidol, in sprayable solution in an intranasal metered dose sprayer. Also provided is a method of administering an antipsychotic drug, such as haloperidol, to a patient, including the step of delivering an effective amount of the antipsychotic drug to a patient intranasally using an intranasal metered dose sprayer. A method of treating a psychotic episode also is provided, the method including the step of delivering an antipsychotic drug, such as haloperidol, intranasally in an amount effective to control the psychotic episode.

### STATEMENT REGARDING FEDERAL SUPPORT

Not Applicable

### BACKGROUND

1. Field of the Invention

Methods and devices for intranasal delivery of antipsychotics, such as haloperidol.

2. Description of the Related Art

A recent focus of antipsychotic drug development for the treatment of acute psychosis is to decrease adverse events related to drug administration, while achieving desired psychiatric control. In the treatment of psychotic episodes, the primary emphasis is to provide both safe and rapid therapy. Currently, the intramuscular route is both the fastest and safest approved method for achieving chemical restraint.

The treatment of patients experiencing psychotic symptoms such as delirium, agitation, and violence is both challenging as well as dangerous for the patient and healthcare provider. This is especially relevant in the emergency room and intensive care settings. In many cases, psychological and behavioral methods for obtaining control are not adequate. It becomes necessary to treat patients using pharmacological avenues that are both safe and rapid. For treatment during acute psychotic episodes, desirable characteristics of an agent include rapid onset, moderate duration of therapeutic action, minimal side effects, predictable bioavailability, ease and safety of administration, and minimal patient discomfort on administration. To date, haloperidol has been the mainstay of treatment for this purpose. To achieve rapid tranquilization, the drug is usually given either intramuscularly or intravenously off-label.

In uncooperative patients, the routes of delivery most commonly used are intravenous (IV) and intramuscular. Although the intravenous route of administration is not FDA approved, it is used for both continuous infusions and bolus injections. For this type of delivery, intravenous access is necessary and the patient should be in an inpatient environment. The intravenous route is commonly used in intensive care settings.

The primary route of administration for uncooperative patients is intramuscular injection. This route is the simplest in uncooperative, aggressive patients. Maximum concentrations can be reached in approximately 20 minutes via this route. Nevertheless, intramuscular administration can be painful and cause a patient already in a psychotic, fragile state of mind to feel even more insecure and increase their sense of being attacked. In addition, muscle enzyme levels may be affected. Both the intravenous and intramuscular routes require the use of needles which poses an infrequent but real risk of infection by blood-borne pathogens to both patients and staff.

If the patient is cooperative, the current desired route of administration is oral. Haloperidol, for example, is available both as a tablet and as an oral solution. The oral solution has a faster onset of action than the tablet and is therefore the dosage form of choice in acute situations. In addition, the oral solution cannot be “cheeked” by patients. One study has shown the onset of action of the oral solution is comparable to that of the intramuscular dose. Patients in these mental states may not readily accept oral medications. However, offering the patient a choice in treatment results in creating a safer environment and restores some sense of autonomy to the patient, which may be particularly helpful when treating psychotic patients. Oral administration of haloperidol results in approximately 65% absolute bioavailability. A wide interindividual variation in the oral absorption of haloperidol has been demonstrated.

Ideally a product should provide both rapid effect and assured dose bioavailability while still allowing the patient to feel included in their treatment and comfortable in their surroundings. This could be accomplished through avoidance of invasive or possibly perceived threatening treatment and delivering the drug in a manner that consistently provides expected, reliable plasma drug concentrations. Optimum treatment such as this necessitates the development of an alternative route of administration.

U.S. Patent Publication No. 2003/0017118A1 discloses an inhaled dosage form for delivery of antipsychotic drugs. Although that publication describes an effective route of administration for antipsychotics for pulmonary inhalation, the delivery of drugs to the lungs often causes coughing and gagging, which can serve to exacerbate the already difficult antipsychotic treatment process. Delivery of medications by oral inhalation is challenging. In standard oral inhalation with a metered dose inhaler, it is common that only 15% of the drug actually makes it to the lung for absorption. Most of the drug formulation impacts in the pharyngeal area and is swallowed. Oral inhalation requires a trained and cooperative patient. Particle size for oral inhalation needs to be less than 10 microns, with optimal particle sizes of less than 3 microns, to be respirable. Further, propellants are commonly used for pulmonary administration.

Haloperidol is an ideal antipsychotic drug for use in emergency situations due to its rapid onset of action, lack of cardiovascular adverse events, decreased respiratory depression, and a low to moderate sedation profile compared to other agents such as chlorpromazine and the benzodiazepines. Haloperidol is associated with a high incidence of extrapyramidal symptoms that can be treated effectively with anticholinergic agents. Newer antipsychotic agents have been developed to minimize the incidence of adverse events while improving treatment. Despite efforts to identify a superior antipsychotic, haloperidol remains the most widely used and cost-effective agent for rapid neuroleptization and is commonly used in studies as a standard measure of efficacy.

Haloperidol, a potent butyrophenone, has been used in clinical practice for more than three decades and is an effective antipsychotic used in a wide variety of psychiatric conditions. Haloperidol is a central dopaminergic receptor antagonist. The first literature descriptions of synthesis and testing of this medication date to the late 1950s.

The structure of haloperidol is given below (Formula I).

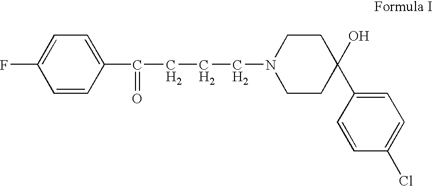

The chemical formulation of haloperidol is C₂₁H₂₃ClFNO₂ with a molecular weight of 375.87. It occurs as a white to faintly yellowish, amorphous or microcrystalline powder. The lactate salt is used to formulate the oral solution and immediate release injectable dosage forms. Lactic acid is used to adjust the injection to a pH of 3-3.8. Haloperidol is well-accepted in the medical community as a safe and effective antipsychotic agent. Typical intramuscular doses range from 2-5 mg/dose in clinical practice. The injection may be repeated as often as every hour although dosing intervals of 4 to 8 hours usually are sufficient. The peak plasma concentration following intramuscular administration occurs at approximately 20 minutes. Haloperidol is metabolized by the liver into inactive metabolites except for reduced haloperidol, which has very limited clinical activity. This metabolism is primarily mediated by the CYP3A4 isoenzyme. The half-life of the lactate formulation in man ranges from 10 to 38 hours.

### SUMMARY

Intranasal delivery of an antipsychotic, such as haloperidol, provides an alternative drug delivery route when parenteral, inhalation or oral administration is unacceptable, undesirable or unwarranted. Rapid systemic availability and the avoidance of hepatic first pass metabolism is possible with this delivery method. This method also avoids generation of biohazardous waste products (needles, blood-contaminated waste, etc.).

In one embodiment, a device is provided for delivering an antipsychotic drug intranasally, comprising an intranasal metered dose sprayer containing one or more doses of a sprayable, aqueous solution including an amount of an antipsychotic drug effective to treat a psychotic episode. The antipsychotic drug can be haloperidol or a salt thereof, such as haloperidol lactate. Other suitable antipsychotic drugs include: butyrophenones, phenothiazines, thiozanthenes, miscellaneous antipsychotics and “new generation” or “atypical” antipsychotics, including: benperidol, droperidol, fluanisone, haloperidol decanoate, moperone chlorohydrate, pipamperone dichlorohydrate, trifluperidol chlorohydrate chlorpromazine, prochlorperazine, fluphenazine, trifluoperazine, perphenazine, acetophenazine, carphenazine, triflupromazine, mesoridazine, thioridazine, thiothixene, chlorprothixene, loxapine, molindone, pimozide, clozapine, risperidone, olanzapine, setindole, supiride, amisulpride and remoxipride.

A method of administering an antipsychotic drug to a patient also is provided. The method includes the step of delivering intranasally an amount of an antipsychotic drug, such as haloperidol lactate, effective to treat a psychotic episode in a sprayable, aqueous solution.

A method of treating a psychotic episode also is provided. The method includes the step of delivering intranasally an effective amount of an antipsychotic drug, such as haloperidol lactate, in a sprayable, aqueous solution.

Lastly, a sprayable solution for use in an intranasal metered dose sprayer is provided. The solution comprises from greater than 0.25% wt. to about 10% wt. haloperidol or a dose equivalent thereof of an antipsychotic drug other than haloperidol, and a pharmaceutically acceptable excipient.

### BRIEF DESCRIPTION OF THE DRAWINGS

FIG. 1 is a graph showing serum concentration levels of haloperidol after intranasal, intravenous and intramuscular drug delivery.

### DETAILED DESCRIPTION

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

Described herein are devices and methods for delivering antipsychotic drugs intranasally, such as haloperidol. The antipsychotic drugs are delivered in aqueous form by a metered dose sprayer.

The terms “antipsychotic,” “antipsychotic agent” and “antipsychotic drug,” and plurals thereof, are synonymous and denote a psychoactive drug that has the ability to calm psychotic states and make a psychotic patient more manageable. A psychotic state may be characterized by psychosis—characterized, without limitation, by gross impairment in reality testing as evidenced by delusions, hallucinations, markedly incoherent speech or disorganized and agitated behavior without apparent awareness on the part of the patient of the incomprehensibility of his behavior. Antipsychotic drugs also are commonly referred to as neuroleptics or neuroleptic drugs. Classes of antipsychotic drugs include butyrophenones, of which haloperidol is the most prominent member, but also include, without limitation, benperidol, droperidol, fluanisone, haloperidol decanoate, moperone chlorohydrate, pipamperone dichlorohydrate and trifluperidol chlorohydrate; phenothiazines, such as chlorpromazine, prochlorperazine, fluphenazine, trifluoperazine, perphenazine, acetophenazine, carphenazine, triflupromazine, mesoridazine and thioridazine; thiozanthenes, such as thiothixene and chlorprothixene; miscellaneous antipsychotics, such as loxapine, molindone and pimozide; and “new generation” or “atypical” antipsychotics, such as clozapine, risperidone, olanzapine, sulpiride, amisulpride, remoxipride and setindole. Of these, haloperidol is preferred in many instances due to its broad acceptance, as indicated above. Other antipsychotics might be preferred in certain instances for any number of reasons, including cost, patient tolerance and efficacy, the degree of sedation caused by the drug, autonomic side effects, such as dry mouth, blurred vision, constipation, urinary retention and reduced blood pressure, and involuntary movement (for example, parkinsonism-like tremors) caused by the drug. Antipsychotics that may be preferred for their limited sedation, autonomic side effects and/or involuntary movement include, without limitation, fluphenazine, perphenazine, thiothixenepimozide, risperidone and olanzapine. Reference to any drug substance herein includes, unless specifically mentioned otherwise, pharmaceutically acceptable salts of that drug substance. For example, the term “haloperidol” refers both to haloperidol and to salts thereof, including, without limitation, haloperidol lactate. All described antipsychotic drugs can be formulated into a sprayable solution according to methods known in the pharmaceutical arts.

An “intranasal” dosage form is a dosage form for delivering an active ingredient to mucosa of the nasal cavity and nasopharynx. Intranasal dosage forms typically introduce particles into the nasal cavity, and the process of inhalation through the nose often is used to facilitate the delivery process. The particles typically are substantially retained in the nasal cavity and nasopharynx because: 1) the particles are introduced through the nose and 2) the particles are sufficiently large that they substantially do not travel past the nasopharynx and into the remainder of the respiratory system. This is in contrast to inhaled dosage forms in which the formulation typically is administered orally, and particle size is gauged to reach the bronchii, the bronchioli and alveoli, as is shown, for illustrative purposes only, in U.S. Patent Publication No. 2003/0017118, in which an aerosolized antipsychotic drug is delivered by the inhalation route.

A “intranasal metered dose sprayer” is a device for repeated delivery of defined quantities of a drug product through the intranasal route. Such devices are broadly available and modes of their use are well-known in the pharmaceutical field. One example of an intranasal metered dose sprayer is described in International Patent Publication No. WO 02/13886, designating the United States, which is incorporated herein by reference in its entirety. The device of WO 02/13886 is programmable and provides individual vials for each dose. Another metered dose sprayer for intranasal use for delivery of multiple single doses of a powder from a single vial is described in U.S. Pat. No. 6,055,979. Lastly, metered dose sprayers for intranasal use are broadly available commercially, for example, from Pfeiffer GmbH of Radolfzell, Germany.

Haloperidol lactate has an accepted dosage range from about 0.1 mg to about 200 mg, or even higher in some cases. Dosing frequency typically ranges from 1 dose per hour to one single dose that is not repeated, as is necessary and effective to manage a patient's symptoms. Tablet formulations available commercially include: 0.5 mg, 1 mg, 2 mg, 5 mg, 10 mg and 20 mg of haloperidol lactate. Liquid oral formulations include 1 mg/mL and 2 mg/mL solutions of haloperidol lactate. Parenteral formulations include 5 mg/mL ( haloperidol lactate), 50 mg/mL (haloperidol decanoate) and 100 mg/mL (haloperidol decanoate) products. Specific intranasal doses for haloperidol lactate include any increment ranging from 0.1 mg to 100 mg, including, without limitation: 0.5 mg, 1 mg, 2 mg, 2.5 mg, 5 mg, 10 mg, 20 mg, 25 mg, 50 mg and 100 mg. In formulating a suitable dosage form for intranasal spraying, the concentration of the drug in the sprayable solution typically is ten times that of an oral or injectable dose in order to achieve satisfactory doses in a typical 0.1 mL spray dose. Thus, a typical intranasal haloperidol solution concentration for a sprayable solution can range between greater than 0.25% wt. to about 10% wt., more typically from about 1.0% wt. to about 10% wt., with 2.5% wt. being the concentration described in the experiments, below.

Haloperidol lactate doses may be calculated based on the weight of the patient, for example, and without limitation, any increment in the range of from about 0.005 mg/kg (weight of patient) to about 10 mg/kg, including, without limitation, 0.01 mg/kg, 0.05 mg/kg, 0.1 mg/kg, 0.5 mg/kg, 1.0 mg/kg, 2 mg/kg, 5 mg/kg or 10 mg/kg. Further, effective serum levels of haloperidol lactate may include any increment in the range of from about 5 mg/L to about 25 mg/L, typically ranging from about 8 mg/L to about 17 mg/L, for example, and without limitation, 8 mg/L, 10 mg/L, 12 mg/L, 15 mg/L and 18 mg/L.

As used herein, in the context of a dosage range, an “increment” can be any value within a range that can be physically delivered in the dosage form. Physical limitations of the intranasal metered dose spray device and in the ability to prepare precisely an intranasal solution with a given concentration of an antipsychotic active ingredient will limit the size of the increments, which include, without limitation, increments of 0.001, 0.01, 0.1 and 1.0 mg, mg/kg or mg/L, as is appropriate.

Dose equivalents for other antipsychotics are well established. In reference to a 2 mg dose of haloperidol lactate, Table A provides non-limiting examples of dose equivalents and suitable doses for certain other antipsychotics.

TABLE A*

| Chemical | Drug Name | Dose equivalent** | Dose range (mg)** | |
|---|---|---|---|---|
| classification | Generic (Trade) | (mg) | (frequency) | |
| Phenothiazine | Chlorpromazine | 100 | 10-100 | mg; q 4-8 h |
| | (Thorazine) | | | |
| | Prochlorperazine | 15 | 2.5-20 | mg; q 4-6 h |
| | (Compazine) | | | |
| | Fluphenazine (Prolixin) | 2 | 0.25-10 | mg; q 6-8 h |
| | Trifluoperazine | 5 | 1-2 | mg; q 4-6 h |
| | (Stelazine) | | | |
| | Perphenazine (Trilafon) | 8 | 4-16 | mg; q 6-12 h |
| | Acetophenazine (Tindal) | 20 | 20-60 | mg; q 24 h |
| | Carphenazine | 25 | 50-150 | mg; q 8-24 h |
| | (Proketazine) | | | |
| | Triflupromazine | 25 | 1-10 | mg; q 6-24 h |
| | (Vesprin) | | | |
| | Mesoridazine (Serentil) | 50 | 25-50 | mg; q 1-8 h |
| | Thioridazine (Mellaril) | 100 | 50-100 | mg; q 6-12 h |
| Thioxanthine | Thiothixene (Navane) | 4 | 2-5 | mg; q 6-12 h |
| | Chlorprothixene | 100 | 10-50 | mg; q 6-8 h |
| | (Taractan) | | | |
| Butyrophenone | Haloperidol (Haldol) | 2 | 0.5-20 | mg; q 0.5-6 h |
| Miscellaneous | Loxapine (Loxitane) | 10 | 10-50 | mg; q 4-6 h |
| | Molindone (Moban) | 10 | 1-75 | mg; q 6-8 h |
| | Pimozide (Orap) | 2 | 1-16 | mg; q 24 h |
| New | Clozapine (Clozapril) | 50 | 25-300 | mg; q 12-24 h |
| Generation | | | | |
| | Risperidone (Risperdal) | 1 | 1-6 | mg; q 12-24 h |
| | Olanzapine (Zyprexa) | 1.5 | 5-20 | mg; q 24 h |
| | Sertindole (Serlect) | 5 | 12-24 | mg; q 24 h |

*See, for example, Table 9.3 of Julien, RM, A PRIMER OF DRUG ACTION, 8^{th} Ed., W.H. Freeman & Co. (1998), p. 259.
**Dose equivalents and dose ranges are approximate. Approximate clinical dose ranges and frequency of administration will differ, depending upon, without limitation, dosage form, patient status/characteristics and titration vs. maintenance.

U.S. Patent Publication No. 2003/0017118 A1 is incorporated herein by reference for its teachings relating to use of various antipsychotic drugs for particulate delivery by the inhalation route and equivalent dosage ranges for those antipsychotics. Dose ranges, whether expressed in terms of mg per dose, mg per kg of patient's weight, or mg/L of patient's serum, plasma or blood, can readily be calculated based on the ratios presented in Table A, or based on any other equivalency data known in the pharmaceutical arts.

In one embodiment of the intranasal dosage forms described herein, the drug is solubilized in water and the formulation therefore is described as “aqueous” which also includes suspensions, emulsions, liposomes, micellar systems and the like, in which the primary solvent is water. In one embodiment, haloperidol is solubilized in a solution of lactic acid. The lactic acid forms the lactate form of haloperidol and adjusts the pH to an acceptable level for intranasal administration. Because haloperidol lactate currently is, for practical reasons, the antipsychotic of choice for uses described herein, use of lactic acid to adjust the pH of the haloperidol solution is preferred. Other compounds can be used, as needed, to adjust, or to buffer the pH of the solution. In the case of haloperidol decanoate, which has low solubility in water, the drug product should include a suitable solubilizing compound or compounds, such as, without limitation: an oil or an ester product thereof, a triglyceride, a cyclodextrin or a surfactant. Many methods for solubilizing water-insoluble drug compounds, such those as certain of the antipsychotics useful in the drug products described herein, are known in the pharmaceutical arts and are appropriate so long as bioavailability of the antipsychotic drug is sufficient for treatment of a patient and adverse reactions, such as irritation to the nasal mucosa, are minimized. In other embodiments, the antipsychotic drug is solubilized in a glycol or in another organic solvents and/or solubilization systems as are known in the pharmaceutical arts.

Other compounds (“excipients”) can be included in the formulation in a manner known in the pharmaceutical arts. Excipients include: acids; bases; buffers; salts; natural and synthetic oils and esters thereof; surfactants; solubilizing compounds, such as cyclodextrins, triglycerides and phospholipids; colorings; flavorings; sweeteners; chelating agents; preservatives; and viscosity modifiers, such as thickening agents. Acids, bases and buffers are collectively referred to herein as “pH modifying agents.” Suitable candidates for any of these agents also are well-known in the pharmaceutical arts.

The formulation also can include a mixture of two or more antipsychotics. The formulation also can include other active ingredients. For instance, the intranasal dosage form might include an anticholinergic agent, such as, without limitation: ipratropium bromide, atropine, scopolamine HBr (Hyoscine HBr), L-hyoscyamine (Anaspaz), L-alkaloids of belladonna (Belafoline), tincture of belladonna alkaloids, homatropine, homatropine methylbromide, methscopolamine (Pamine), anisotropine (Valpin), anisotropine with phenobarbital, clindinium (Quarzan), glycopyrrolate (Robinul), hexocyclim (Tral), isopropamide (Darbid), mepenzolate (Cantil), methantheline (Banthine), oxyphencyclimine (Daricon), propantheline (Pro-Banthine), tridihexethyl (Pathilon), dicyclomine (Bentyl), cyclopentolate (Cyclogyl), tropicamide (Mydriacyl), trihexyphenidyl (Artane), benztropine (Cogentin), orphenadrine HCl (Disipal), ethopropazine (Parsidol), diphenhydramine (Benadryl), cycrimine (Pagitane), biperiden (Akineton) benztropine, benzhezol, procyclidine and orphenadrine to reduce extrapyramidal symptoms.

### EXAMPLE 1

### Haloperidol Intranasal Formulation—25 mg/mL

An intranasal drug product containing haloperidol is manufactured according to the following protocol. 1.18 grams of 85% USP lactic acid is added to approximately 50 g water for injection, USP and mixed. 2.5 grams of haloperidol is slowly added to the lactic acid solution while mixing until the haloperidol is completely dissolved. The solution is then brought to a volume of 100 mL, and is then filtered (22μ) and autoclaved. The solution then is dispensed in appropriate amounts into an intranasal metered dose sprayer to deliver a desired dose of haloperidol, such as 100 μL (2.5 mg). For any metered dose sprayer, such as that of International Patent Publication WO 02/13886, the amount dispensed is that dosage amount plus a residual volume of solution that cannot be dispensed by the delivery device.

### EXAMPLE 2

### Antipsychotic Formulations

Additional intranasal formulations may be prepared as follows. Table B provides suitable ranges for these additional products.

TABLE B
additional antipsychotic compounds and ranges.

| | Concentration | | |
|---|---|---|---|
| | Range for Drug | Dosage range | |
| Drug substance | Product (mg/mL) | (per dose) | Excipient |
| Haloperidol | 10 to 100 | 0.5 mg to 100 mg | Lactic acid |
| Risperidone* | 1 to 200 | 0.1 to 20 | |
| Olanzapine* | 50 to 200 | 5 to 20 | |

*In a mildly acidic aqueous system or a non-aqueous system

Additional ingredients may be added to the formulations described above, including, sweeteners, flavors, colorings, buffers, salts, chelating agents, preservatives, and viscosity modifiers.

### EXAMPLE 3

### Bioavailability Study Comparing Intranasal, Intramuscular and Intravenous Administration of Haloperidol Lactate

The following compares the bioavailability of 2.5 mg haloperidol delivered intranasally, intramuscularly and intravenously. The intranasal dosage form is described in Examples 1 and 2, above. One spray containing 2.5 mg haloperidol in 100 μL was administered to one nostril for each patient. The intramuscular dose was given as a single injection of 0.5 mL of a 5.0 mg/mL solution of haloperidol lactate (Haloperidol Injection (5.0 mg/mL) by Ortho-McNeil). The intravenous dose was prepared by diluting 0.5 mL of a 5.0 mg/mL solution of haloperidol lactate in D₅W (5% dextrose in water) and was infused over 15 minutes.

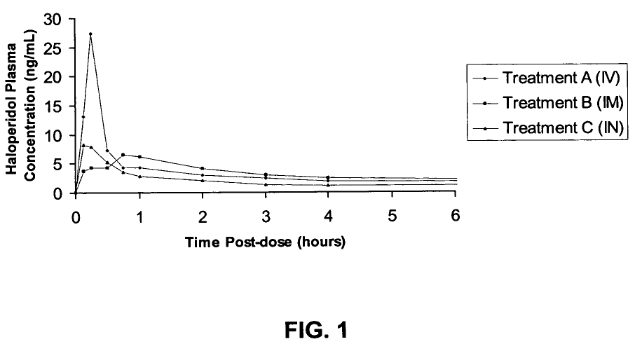

The pharmacokinetic results of the study are as follows. The mean (n=4; 2 male, 2 female) plasma concentration versus time curve profiles over the first 4 hours is shown in FIG. 1. This FIGURE shows that absorption of haloperidol following intranasal administration was very rapid. Haloperidol concentrations reached a peak in one subject at 7.5 minutes, in two individuals at 15 minutes, and at 30 minutes for the last subject.

Table C summarizes pharmacokinetic data for the three treatments. Median Tₘₐₓ values were 15 and 37.5 minutes for the intranasal and intramuscular doses, respectively. One subject had an unexpected Tₘₐₓ of 16 hours following a gradual increase in plasma concentration over time after intramuscular administration. Cₘₐₓ values after the intranasal dose were slightly higher than values obtained after the intramuscular dose and occurred consistently earlier. One subject had a Cₘₐₓ of 16.2 ng/mL following intranasal administration. The other subjects had Cₘₐₓ values between 7.5-7.9 ng/mL after intranasal delivery.

Relative bioavailability of the intramuscular dose to intranasal dose was on average 48.6%. The absolute bioavailability of haloperidol by the intranasal and intramuscular routes was 63.8% and 134.5%, respectively. Clearance terms were comparable between the administration routes. Intravenous and intranasal had similar half-lives.

TABLE C
Mean (CV as a %) single dose haloperidol lactate pharmacokinetic (PK)
parameters following administration of 2.5 mg intravenous, intramuscular,
and intranasal haloperidol lactate in healthy subjects (n = 4).

| | IV | IM | IN |
|---|---|---|---|
| PK Parameter | (2.5 mg) | (2.5 mg) | (2.5 mg) |
| Tₘₐₓ (h)* | 0.25 (0.25-0.25) | 0.625 (0.25-16) | 0.25 (0.12-0.5) |
| Cₘₐₓ (ng/mL) | 23.3 (65.37) | 8.42 (91.41) | 9.8 (43.57) |
| t_{1/2} (h) | 17.1 (45.5) | 28.8 (19.1) | 16.9 (45.0) |
| AUC₀₋ₜ | 50.77 (16.78) | 59.69 (29.65) | 31.67 (14.84) |
| (ng · h/mL) | | | |
| AUC_{0-∞} | 60.4 (15.6) | 81.8 (26.7) | 37.9 (21.5) |
| (ng · h/mL) | | | |
| MRT (h) | 12.7 (29.6) | 16.1 (35.3) | 12.9 (22.7) |
| CL/F or CLₛₛ/ | 42.1 (15.5) | 32.8 (33.9) | 68.3 (20.3) |
| F(L/h) | | | |
| Vₛₛ (L) | 934.0 (44.9) | — | — |
| V_{z}/F (L) | 1069 (51.5) | 1356 (38.7) | 1575 (39.4) |
| F (%) | assume 100% | 134.5 (20.8) | 63.8 (24.4) |
| Relative F | — | — | 48.6 (29.4) |
| (IM/IN) (%) | | | |

*median and range given for Tₘₐₓ

Thus, this pilot study demonstrated that haloperidol is bioavailable by this route in clinically meaningful concentrations. These data demonstrate that haloperidol lactate is effectively delivered through the intranasal route, thus providing a safe and non-threatening alternative to injection or inhalation of haloperidol.

Whereas particular embodiments of the invention have been described herein for the purpose of illustrating the invention and not for the purpose of limiting the same, it will be appreciated by those of ordinary skill in the art that numerous variations of the details, materials and arrangement of parts may be made within the principle and scope of the invention without departing from the invention as described in the appended claims.

We claim:

1. A device for delivering an antipsychotic drug intranasally, comprising an intranasal metered dose sprayer containing one or more doses of a sprayable solution comprising an amount of an antipsychotic drug effective to treat a psychotic episode.

2. The device of claim 1, wherein the antipsychotic drug is haloperidol.

3. The device of claim 2, wherein the sprayable solution comprises an amount of haloperidol ranging from between about 10 mg/mL and about 100 mg/mL.

4. The device of claim 3, wherein the sprayable solution contains about 25 mg/mL haloperidol.

5. The device of claim 2, wherein the sprayable solution further comprises a pH modifying agent.

6. The device of claim 5, wherein the pH modifying agent is lactic acid.

7. The device of claim 2, wherein the sprayable solution comprises a lactate salt of haloperidol.

8. The device of claim 2, wherein the dose of haloperidol ranges from between 1.0 mg and 10 mg.

9. The device of claim 8, wherein the dose is about 2.5 mg haloperidol.

10. The device of claim 2, wherein the sprayable solution is prepared by dissolving haloperidol in a solution of lactic acid.

11. The device of claim 10, wherein, for each mL of sprayable solution, about 25 mg haloperidol is dissolved in a solution containing about 11.8 mg lactic acid.

12. The device of claim 10, wherein the haloperidol is dissolved in a solution containing an amount of lactic acid sufficient to adjust the pH of the sprayable solution to a pH range from about 3.2 to about 5.0.

13. The device of claim 10, wherein the solution of lactic acid comprises an amount of lactic acid ranging between about 0.1% wt. and about 10% wt. and from about 0.1% wt. to about 10% of haloperidol dissolved in the solution of lactic acid, wherein % wt. refers to the mass of the final sprayable, aqueous solution.

14. The device of claim 13, wherein the solution of lactic acid contains about 1% wt. lactic acid and about 2.5% wt. haloperidol dissolved in the solution of lactic acid.

15. The device of claim 1, wherein the intranasal metered dose sprayer comprises a plurality of doses.

16. The device of claim 15, wherein the intranasal metered dose sprayer comprises a plurality of dosage compartments, each compartment containing a single dose of the antipsychotic drug.

17. The device of claim 1, wherein the antipsychotic drug is selected from the group consisting of haloperidol, benperidol, droperidol, fluanisone, haloperidol decanoate, moperone chlorohydrate, pipamperone dichlorohydrate, trifluperidol chlorohydrate chlorpromazine, prochlorperazine, fluphenazine, trifluoperazine, perphenazine, acetophenazine, carphenazine, triflupromazine, mesoridazine, thioridazine, thiothixene, chlorprothixene, loxapine, molindone, pimozide, clozapine, risperidone, olanzapine, setindole, supiride, amisulpride and remoxipride.

18. A method of treating a psychotic episode, comprising the step of delivering intranasally an amount of an antipsychotic drug effective to treat the psychotic episode in a sprayable solution.

19. The method of claim 18, wherein the antipsychotic drug is haloperidol.

20. The method of claim 19, wherein the sprayable solution comprises an amount of haloperidol ranging from between about 10 mg/mL and about 100 mg/mL.

21. The method of claim 20, wherein the sprayable solution contains about 25 mg/ml haloperidol.

22. The method of claim 19, wherein the sprayable solution further comprises a pH modifying agent.

23. The method of claim 22, wherein the pH modifying agent is lactic acid.

24. The method of claim 19, wherein the sprayable solution comprises a lactate salt of haloperidol.

25. The method of claim 19, wherein the dose of haloperidol ranges from between 1.0 mg and 10 mg.

26. The method of claim 25, wherein the dose is about 2.5 mg haloperidol.

27. The method of claim 19, wherein the sprayable solution is prepared by dissolving haloperidol in a solution of lactic acid.

28. The method of claim 27, wherein, for each mL of aqueous solution, about 25 mg haloperidol is dissolved in a solution containing about 11.8 mg lactic acid.

29. The method of claim 27, wherein the haloperidol is dissolved in a solution containing an amount of lactic acid sufficient to adjust the pH of the aqueous solution to a pH range from about 3.2 to about 5.0.

30. The method of claim 27, wherein the solution of lactic acid comprises an amount of lactic acid ranging between about 0.1% wt. and about 10% wt. and from about 0.1% wt. to about 10% wt. of haloperidol dissolved in the solution of lactic acid, wherein % wt. refers to the mass of the fluid sprayable solution.

31. The method of claim 30, wherein the solution of lactic acid contains about 1% wt. lactic acid and about 2.5% wt. of haloperidol dissolved in the solution of lactic acid.

32. The method of claim 18, wherein the antipsychotic drug is delivered by a metered dose sprayer comprising a plurality of doses.

33. The method of claim 32, wherein the intranasal metered dose sprayer comprises a plurality of dosage compartments, each compartment containing a single dose of the antipsychotic drug.

34. The method of claim 18, wherein the antipsychotic drug is selected from the group consisting of haloperidol, benperidol, droperidol, fluanisone, haloperidol decanoate, moperone chlorohydrate, pipamperone dichlorohydrate, trifluperidol chlorohydrate chlorpromazine, prochlorperazine, fluphenazine, trifluoperazine, perphenazine, acetophenazine, carphenazine, triflupromazine, mesoridazine, thioridazine, thiothixene, chlorprothixene, loxapine, molindone, pimozide, clozapine, risperidone, olanzapine, setindole, supiride, amisulpride and remoxipride.

35. A method of administering an antipsychotic drug to a patient, comprising the step of delivering intranasally an amount of an antipsychotic drug effective to treat a psychotic episode in a sprayable solution.

36. The method of claim 35, wherein the antipsychotic drug is haloperidol.

37. The method of claim 36, wherein the sprayable solution comprises an amount of haloperidol ranging from between about 10 mg/mL and about 100 mg/mL.

38. The method of claim 37, wherein the sprayable solution contains about 25 mg/mL haloperidol.

39. The method of claim 36, wherein the sprayable solution further comprises a pH modifying agent.

40. The method of claim 39, wherein the pH modifying agent is lactic acid.

41. The method of claim 36, wherein the sprayable solution comprises a lactate salt of haloperidol.

42. The method of claim 36, wherein the dose of haloperidol ranges from between 1.0 mg and 10 mg.

43. The method of claim 42, wherein the dose is about 2.5 mg haloperidol.

44. The method of claim 36, wherein the sprayable solution is prepared by dissolving haloperidol in a solution of lactic acid.

45. The method of claim 44, wherein, for each mL of sprayable solution, about 25 mg haloperidol is dissolved in a solution containing about 11.8 mg lactic acid.

46. The method of claim 44, wherein the haloperidol is dissolved in a solution containing an amount of lactic acid sufficient to adjust the pH of the sprayable solution to a pH range from about 3.2 to about 5.0.

47. The method of claim 44, wherein the solution of lactic acid comprises an amount of lactic acid ranging between about 0.1% wt. and about 10% wt. and from about 0.1% wt. to about 10% wt. of haloperidol dissolved in the solution of lactic acid, wherein % wt. refers to the mass of the final sprayable solution.

48. The method of claim 47, wherein the solution of lactic acid about 1% wt. lactic acid and about 2.5% wt. haloperidol dissolved in the solution of lactic acid.

49. The method of claim 35, wherein the antipsychotic drug is delivered by a metered dose sprayer comprising a plurality of doses.

50. The method of claim 49, wherein the intranasal metered dose sprayer comprises a plurality of dosage compartments, each compartment containing a single dose of the antipsychotic drug.

51. The method of claim 35, wherein the antipsychotic drug is selected from the group consisting of haloperidol, benperidol, droperidol, fluanisone, haloperidol decanoate, moperone chlorohydrate, pipamperone dichlorohydrate, trifluperidol chlorohydrate chlorpromazine, prochlorperazine, fluphenazine, trifluoperazine, perphenazine, acetophenazine, carphenazine, triflupromazine, mesoridazine, thioridazine, thiothixene, chlorprothixene, loxapine, molindone, pimozide, clozapine, risperidone, olanzapine, setindole, supiride, amisulpride and remoxipride.

52. A sprayable solution for use in an intranasal metered dose sprayer, comprising
a) from greater than 0.25% wt. to about 10% wt. haloperidol or a dose equivalent thereof of an antipsychotic drug other than haloperidol; and
b) a pharmaceutically acceptable excipient.

53. The sprayable solution of claim 52, comprising from greater than 0.25% wt. to about 10% wt. haloperidol lactate.

54. The sprayable solution of claim 52, consisting essentially of from greater than 0.25% wt. to about 10% wt. haloperidol lactate.

55. The sprayable solution of claim 52, comprising about 2.5% wt. haloperidol lactate.
